(19) United States
(12) Patent Application Publication
(10) Pub. No.: US 2026/0007599 A1
(43) Pub. Date: Jan. 8, 2026

(54) TICAGRELOR IV FORMULATIONS FOR USE IN THE TREATMENT OF GRAM-POSITIVE BACTEREMIA

(71) Applicant: Hyloris Developments SA, Liège (BE)

(72) Inventor: Sabine Eeckhaoudt, Liège (BE)

(21) Appl. No.: 19/328,114

(22) Filed: Sep. 13, 2025

Related U.S. Application Data
(63) Continuation of application No. PCT/EP2024/056382, filed on Mar. 11, 2024.
Application No. PCT/EP2024/056382 is a continuation of application No. PCT/EP2023/056322, filed on Mar. 13, 2023.

(51) Int. Cl. A61K 9/08; A61K 9/00; A61K 31/519; A61K 35/19; A61K 45/06; A61K 47/69

(52) U.S. Cl. CPC A61K 9/08; A61K 9/0019; A61K 31/519; A61K 35/19; A61K 45/06; A61K 47/6951

(57) ABSTRACT

The present invention relates to aqueous ticagrelor solutions provided for intravenous administration for use in the treatment of a Gram-positive bacterial infection in the blood stream of a patient in need thereof. The invention is advantageous to further improve the effectiveness of bacteremia treatments, especially for MSSA and MRSA bacteremia and bacteremia-associated thrombocytopenia.

### TECHNICAL FIELD

The present invention is situated in the field of pharmaceutical compositions and medical uses of pharmaceutical compositions. The invention is advantageous as it provides ticagrelor iv solutions, with adequate ticagrelor solubility and storage stability, for use in the treatment of a Gram-positive bacterial bloodstream infection (bacteremia) in an inflicted patient. The present invention improves the effectiveness of medical treatments and may help save lives.

### BACKGROUND

The active ingredient ticagrelor is well-known. Its chemical name is (1S,2S,3R,5S)-3-{7-[(1R,2S)-2-(3,4-difluorophenyl)cyclopropylamino]5-(propylthio)-3H-(1,2,3) triazolo (4,5-D) pyrimidin-3-yl}-5-(2 hydroxyethoxy) cyclopentane-1,2-diol. Ticagrelor has the following chemical structure:

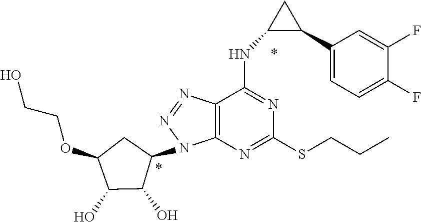 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor has a well-established use in cardiology. It is used in patients with acute coronary syndromes for the prevention of thrombotic events, such as myocardial infarctions or strokes.

Recently, a non-cardiology use for ticagrelor was proposed. Lancelloti et al. reported ticagrelor to have antibiotic properties, ex-vivo, at concentrations that are much higher than those encountered in the treatment of a cardiologic condition (JAMA Cardiol. 2019, 4 (6): 596-599). The minimal bactericidal concentration was 20 microgram/ml against Methicillin sensitive Staphylococcus aureus (MSSA), glycopeptide intermediate Staphylococcus aureus (GISA), Methicillin resistant Staphylococcus aureus (MRSA) and vancomycin-resistant E. Faecalis (VRE); 30 microgram/ml against methicillin-resistant Staphylococcus epidermis (MRSE); and 40 microgram/ml against Enterococcus faecalis and Staphylococcus agalactiae.

In WO2018046174, Lancelotti and Oury disclosed the use of triazolo (4,5-D) pyrimidine derivatives, for treatment of bacterial infection. Ticagrelor is part of a long list of defined triazolo (4,5-D) pyrimidine derivatives (FIG. 1). It is disclosed that the triazolo (4,5-D) pyrimidine derivatives of the invention may exhibit effect through different types of formulations. However, specific ticagrelor compositions or ticagrelor use in a treatment of a bloodstream infection were not disclosed.

Ulloa et al. (J Infect Dis. 2021, 224 (9): 1566-1569) reported the first experimental use of ticagrelor in a single human patient for the treatment of a bacterial infection with Staphylococcus aureus bacteria. Ticagrelor tablets of 90 mg were administered orally twice daily from day 5 onwards, in addition to antibiotic treatment with cefazolin plus ertapenem. In total, ticagrelor tablets were taken for a total of 3 months. The patient spent 35 days in hospital.

Although the outcome was very promising, the use of tablets may not be acceptable in routine clinical practice, since several countries have guidelines that dictate the use of intravenous formulations for the treatment of a bacteremia, especially in case of a deep-seated bacterial infection that cannot be surgically removed (complicated bacteremia). Tablets and other means of oral intake have the disadvantage that bioavailability is limited. Oral bioavailability of ticagrelor is 36% (30-42% confidence interval), with large variability between individuals (Teng and Maya, J Drug Assess. 2014, 3 (1): 43-). Consequently, there is a need for an improved formulation, especially in an emergency or life-threatening situation.

It is generally known that intravenous administration works faster (shorter half-life), bioavailability is 100% and variability is less than for oral. It would be beneficial to have a ticagrelor formulation suitable for intravenous administration; preferably in a ready-to-use formulation. However, ticagrelor is notoriously difficult to handle. The active ingredient is susceptible to degradation when exposed to light, heat and oxygen. In addition, its limited solubility is a great challenge in the development of pharmaceutical formulations. Ticagrelor has a low and pH-independent solubility in aqueous media. This property of not ionizing in the physiological pH range, makes the development of liquid formulations especially challenging. Yaye et al. (J Pharm Biomed Anal. 2015; 105:74-83) studied the degradation of ticagrelor when exposed to heat, pH, peroxide and light. They identified numerous degradants DPI to DP9 indicating that the molecule is highly suseptible to degradation.

Ticagrelor is commercially available in the form of tablets. 60 and 90 mg tablets are available under the tradename Brilinta® in the US and under the tradename Brilique® in EU. Ticagrelor is not commercially available in liquid form.

In the product characteristics for the medicinal product Brilique 60 mg or 90 mg film-coated tablets (Brilique INN-ticagrelor) it is disclosed that tablets can be crushed, mixed with water and drunk immediately, for patients who are unable to swallow tablets. Alternatively, the mixture may be administered via a nasogastric tube into the stomach. The disadvantage of this formulation is that it is not readily available to patients. A preparation is needed just prior administration. The formulation does not have a long-term storage stability and the tablet particles settle on standing, which excludes the formulation for intravenous administration. In addition, this formulation is not sterile.

Sigfridsson et al. (J Pharm Sci. 2011, 100 (6): 2194-2202) disclosed a composition based on nanoparticles of ticagrelor, a combination of polyvinylpyrrolidone and the disodium salt of Aerosol AOT for stabilization of the active ingredient, and 5 percent mannitol to obtain a nanosuspension. Aerosol AOT is believed to correspond to dioctyl sulfosuccinate sodium salt. Although the nanosuspension is reported to have a stability of at least 10 months, it is also reported that there is some tendency for particle aggregation and sedimentation during storage. Therefore, samples should be sonicated prior to an intravenous administration. This is cumbersome for a pharmaceutical use and presents a safety risk. Especially in a situation requiring an acute treatment, this formulation is not robust for hospital use.

The same publication of Sigfridsson et al. also mentions that ticagrelor concentrations in a phosphate buffered solution at pH 7.4 declined after 1 month under normal laboratory conditions of light and temperature.

Na et al. (Int J Nanomedicine. 2019, 14:1193-1212) studied self-micro emulsifying drug delivery systems (SMEDDS) for oral delivery to overcome the poor ticagrelor solubility barriers. Ticagrelor solubility was studied in oily and hydrophilic excipients. A combination of surfactants was selected to obtain an emulsion system: Capmul MCM/Cremophor EL/Transcutol P. However, oily substances and surfactants are deemed not suitable for use in a formulation for intravenous administration.

Teng and Maya disclosed the use of a single iv infusion of ticagrelor 15 mg (150 ml of ticagrelor solution for infusion: 0.1 mg/ml given at 300 ml/h over 30 min) in healthy volunteers (J Drug Assess. 2014, 3 (1): 43-50). No information is provided on the composition or stability. Presumably it was a freshly prepared composition to allow a comparison in pharmacokinetics of oral and iv administration. A use in patients was not reported.

In view of the above, there remains a need in the art for ticagrelor iv formulations for use in the treatment of a Gram-positive bacterial infection in the bloodstream of an inflicted patient.

The objective of the present invention is to solve at least one or more problems as described above. In particular, the invention aims to provide ticagrelor iv formulations for use in the treatment of a Gram-positive bacteremia. Ticagrelor iv should be readily available to patients and the formulation preferably makes use of ingredients that regulatory agencies find acceptable (e.g. within the FDA's Inactive Ingredient Guide-IIG limits) to enable market introduction and patient availability.

### SUMMARY OF THE INVENTION

In a first aspect, the invention provides a pharmaceutical ticagrelor composition for use in the treatment of a Gram-positive bacteremia in a patient in need thereof by administration of the pharmaceutical composition in a therapeutically effective concentration, characterized in that the pharmaceutical ticagrelor composition is an aqueous ticagrelor solution, the pharmaceutical ticagrelor composition has a storage stability of at least 3 months at 25° C. and 60% Relative Humidity or at 40° C. and 75% Relative Humidity, and administration is intravenously, either by injection or infusion.

Availability of a ticagrelor iv composition with adequate ticagrelor solubility and limited degradation for use in the treatment of a Gram positive bacteremia infection in advantageous as intravenous administration provides 100% bioavailability, allows easier dose adjustment and can be applied even when a patient is unconscious. Intravenous formulations are advantageous for use in the treatment of deep-seated bacterial infections that cannot be surgically removed.

Preferably, the pharmaceutical ticagrelor composition has a pH of 5,5 to 9,0 and an osmolality between 300-900 mOsm/kg.

Preferably, the pharmaceutical ticagrelor composition comprises a water-soluble inclusion complex of ticagrelor in a cyclodextrin; preferably the cyclodextrin is a hydroxypropyl-beta-cyclodextrin.

Preferably, organic co-solvents are excluded.

Preferably, the pharmaceutical ticagrelor composition comprises 1-15 mg/ml ticagrelor and 15-40% w/w cyclodextrin.

Preferably, the pharmaceutical ticagrelor composition has a volume of 1-15 ml for administration by injection or having a volume of 10-50 ml for administration by short-term infusion of at most 30 minutes.

Preferably, the pharmaceutical ticagrelor composition comprises 0,10-14 mg/ml ticagrelor and 20-100 mg/ml of cyclodextrin.

Preferably, the pharmaceutical ticagrelor composition has a volume of 25 to 1000 ml for administration by infusion for at least 30 minutes.

Preferably, the Gram-positive bacteremia is a Staphylococcus, Streptococcus or Enterococcus bacteremia; preferably the Gram-positive bacteremia is a Staphylococcus aureus bacteremia; more preferably the Gram-positive bacteremia is an antibiotic resistant bacterial infection; most preferably the Gram-positive bacteremia is a methicillin-sensitive (MSSA) or methicillin-resistant Staphylococcus aureus (MRSA).

Preferably, the patient is further administered an antibiotic selected from cefazolin, ceftaroline, daptomycin, ertapenem, linezolid, minocycline, oxacillin, telavancin, trimethoprim-sulfamethoxazole, vancomycin or combinations thereof.

Preferably, intravenous administration of ticagrelor is initiated within 4 days of the bacterial blood stream infection.

Preferably, the dose of ticagrelor is adjusted in accordance with the alpha toxin levels produced by the Gram-positive bacterial strain present in the blood stream infection.

Preferably, the patient has a blood platelet count between 50.000 and 150.000 platelets per microliter of blood (thrombocytopenia).

Preferably, the treatment further comprises the administration of an effective amount of blood platelets for the treatment of the thrombocytopenia.

Preferably, the blood platelets are pre-treated with ticagrelor.

In another aspect, the invention provides a container comprising a pharmaceutical ticagrelor composition according to the first aspect.

Preferably, the container is a plastic bag or glass bottle.

In further aspects, the invention provides ready-to-use ticagrelor products for use in the treatment of a Gram-positive bacteremia in a patient in need thereof by administration of the pharmaceutical composition in a therapeutically effective concentration, wherein administration is intravenously by infusion.

Preferably, ready-to-use ticagrelor products are a ready-to-use container comprising 25-1000 ml of an aqueous solution, 0,10-14 mg/ml ticagrelor and 20-100 mg/ml of a cyclodextrin; preferably a hydroxypropyl-beta-cyclodextrin.

It is beneficial to have a ready-to-use iv formulation, especially for use in the treatment of severe bacteremia, which may lead to life-threatening thrombocytopenia (loss of blood platelets). Also, patients may be unconscious and not capable of taking tablets.

### DETAILED DESCRIPTION OF THE INVENTION

Unless otherwise defined, all terms used in the description of the invention, including technical and scientific terms, have the meaning as commonly understood by a person skilled in the art to which the invention pertains. Furthermore, definitions of the terms are included to better understand the description of the present invention.

As used here, the following terms have the following meaning:

“A”, “an”, and “the” as used here refer to both the singular and the plural, unless the context indicates otherwise. “A surfactant” refers, by way of example, to one or more than one surfactant.

“About” as used herein, referring to a measurable value such as a parameter, an amount, a duration and the like, is intended to include variations of plus or minus 10% or less, preferably plus or minus 5% or less, more preferably plus or minus 3% or less, even more preferably plus or minus 1% or less, and even more preferably plus or minus 0.1% or less of the specified value, as far as such variations are suitable for carrying out in the described invention. It will be clear, however, that the value to which the term “about” relates is itself also specifically described. “Include”, “comprising” and “comprises” used herein are synonymous with “contain”, “containing” or “contains” and are inclusive or open terms that specify the presence of what follows, e.g. a component and the presence of additional, unnamed components, features, elements, parts, steps, which are well known in the art or described therein, and do not exclude them.

The recitation of numerical ranges by endpoints includes all numbers and fractions that are included within that range, as well as the endpoints mentioned.

The term “% w/w” as used herein means percentage by weight in which the weight ratio of an ingredient to the total weight of a composition is expressed as a percentage.

Bacterial bloodstream infections, called bacteremia, are notoriously difficult to treat especially when they are caused by Gram-positive bacteria such as methicillin resistant Staphylococcus aureus strains. Methicillin resistant Staphylococcus aureus strains give of large amounts of alpha toxins. These toxins attack the blood platelets, rendering them insufficient to trigger the immune system to fight of the bacterial infection. In addition, large amounts of blood platelets may be damaged and lost (thrombocytopenia). This leads to an increased risk of bleeding and life-threatening situations (Alhurayri et al. Toxins, 2021, 13 (10): 726).

The standard method of care of a bacterial infection in the blood stream is a treatment with antibiotics. For hospital-incurred bacteremia infections, intravenous formulations are used. Intravenous formulations have the benefit of a 100% bioavailability over oral medication. They allow a rapid onset of the treatment. Intravenous formulations are sought after especially for the treatment of bloodstream infections that are caused by a bacterial infection source that is not surgically removable and hard to reach.

Recently, ticagrelor tablets have been put forward for consideration in the treatment of bacteremia infections. Ticagrelor tablets however have a bioavailability of only 36%, with a large between person variability. Intravenous formulations are thus far not commercially available. Past development efforts have failed to solubilize ticagrelor in concentrations that are medically relevant. In addition, the limited storage stability was inadequate to move beyond freshly made compositions.

The present invention provides aqueous ticagrelor solutions that are provided for intravenous administration. Suitable aqueous ticagrelor solutions for use in the present invention are described in co-pending applications PCT/EP2022/063185 and PCT/EP2023/055736 which are herein enclosed by reference.

The aqueous ticagrelor solutions used in the invention have adequate storage stability, which makes them suitable for commercialization. Without adequate storage stability, it would not be possible to produce the solution, package it, store it, transport to the site of use, store until required and use it in the treatment of patients. An adequate storage stability that can avoid solutions being prepared and used on demand, is believed to require a storage stability of at least 3 months as measured under storage stability conditions of 25° C. and 60% Relative Humidity or under accelerated storage stability conditions of 40° C. and 70% Relative Humidity.

More preferably said storage stability is at least 6 months; even more preferably at least 9 months; most preferably at least 12 months. A satisfactory stability of 6 months at 40° C. and 75% RH corresponds to a shelf life of 24 months at room temperature of 25° C.

“Storage stability” as used herein means that the total impurity level is below 0.5%.

The aqueous ticagrelor solutions can advantageously be used in the treatment of a Gram-positive bacteremia by intravenous administration of an effective amount of the aqueous ticagrelor solution to a patient in need thereof. Depending on the concentration of ticagrelor, the intravenous administration is by injection or infusion.

The invention provides a solution to the problem of treating a Gram-positive bacteremia with ticagrelor in an improved way.

In particular, the invention provides a pharmaceutical ticagrelor composition for use in the treatment of a Gram-positive bacteremia in a patient in need thereof by administration of the pharmaceutical composition in a therapeutically effective concentration, characterized in that the pharmaceutical ticagrelor composition is an aqueous ticagrelor solution, the pharmaceutical ticagrelor composition has a storage stability of at least 3 months at 25° C. and 60% Relative Humidity or at 40° C. and 75% Relative Humidity, and administration is intravenously, either by injection or infusion.

With the term “bacteremia” as used herein, is meant a bacterial infection in the bloodstream.

With the term “Gram-positive bacteria” as used herein, is meant bacteria that give a positive result in the Gram stain test, which is a test known to the person skilled in the art to classify bacteria according to their type of cell wall. Gram-positive bacteria take up the crystal violet stain used in the test and appear to be purple-coloured when seen through an optical microscope. This is because the thick peptidoglycan layer in the bacterial cell wall retains the stain after it is washed away from the rest of the sample, in the decolorization stage of the test. Conversely, gram-negative bacteria cannot retain the violet stain after the decolorization step; alcohol used in this stage degrades the outer membrane of gram-negative cells, making the cell wall more porous and incapable of retaining the crystal violet stain. Their peptidoglycan layer is much thinner and sandwiched between an inner cell membrane and a bacterial outer membrane, causing them to take up the counterstain, such as safranin or fuchsine, and appear red or pink.

In a preferred embodiment the bacteremia is a Staphylococcus, Streptococcus or Enterococcus bacteremia. Preferably the bacteremia is a Staphylococcus aureus bacteremia. More preferably the bacteremia is an antibiotic resistant bacterial infection with Gram-positive bacteria. Most preferably the bacteremia is a methicillin-sensitive (MSSA) or methicillin-resistant Staphylococcus aureus (MRSA) bacteremia. MRSA bacteremia is notoriously difficult to treat. The mortality rate is at least 20%.

The patient is preferably a mammal, more preferably a human.

The invention is advantageous as it makes storage stable liquid formulations of ticagrelor available, which are suitable for administration into a vein. Veins are tubes forming part of the blood circulation system of the patient's body, carrying blood and blood platelets, also called thrombocytes. The use of a liquid formulation provided for intravenous administration in a bacteremia treatment improves the bioavailability of ticagrelor to blood platelets in the bloodstream thereby providing protection against bacterial attack. As blood platelets are important in the immune system, their protection is deemed important to help fight of a Gram-positive bacterial blood stream infection.

Preferably the pH of the product is such that a pH-adjustment prior to administration is not required. The aqueous ticagrelor solution as described above preferably has a pH of 5,5 to 9,0 more preferably 6,0 to 8,5, even more preferably 6,5 to 8,0, most preferably 6,8 to 7,8.

Preferably the osmolality of the product is such that an adjustment prior to administration is not required. The aqueous ticagrelor solution as described above, used in the present invention, preferably has an osmolality between 300 and 900 mOsm/kg; more preferably between 400 and 850 mOsm/kg, most preferably between 450 and 800 mOsm/kg. An osmolality between 300-900 mOsm/kg allows intravenous administration without prior adjustments of the formulation. This osmolality is compatible with a direct iv administration of the ticagrelor formulation in a bacteremia treatment. These parameters are beneficial for compatibility with the bloodstream of a patient.

In a preferred embodiment, the pharmaceutical ticagrelor composition further comprises a buffering agent. The buffering agent is preferably a phosphate buffer. More preferably the aqueous ticagrelor solution for use in the present invention has a 5 mM-20 mM phosphate buffer. This buffer strength was found to be advantageous for obtaining the desired storage stability of the aqueous ticagrelor solution.

The ticagrelor in a composition according to the invention is solubilized using a solubilizing agent. Preferably the solubilizing agent is a cyclodextrin. Preferably the cyclodextrin is a hydroxypropyl-beta-cyclodextrin. Cyclodextrin, especially hydroxypropyl-beta-cyclodextrin, was found to provide a water-soluble inclusion complex of ticagrelor.

Preferably the aqueous pharmaceutical ticagrelor solution according to an embodiment of the invention has a volume of 5-1000 ml. This volume spans the range of several types of intravenous administration, wherein small volumes are typically administered by bolus injection. Larger volumes are administered by infusion.

The aqueous ticagrelor solution for intravenous administration, preferably lack any other solvent or surfactant. In particular, co-solvents comprised of oil, ethanol, propylene glycol, polyethylene glycol are excluded. Preferably also polymers comprised of poloxamers, polyvinylpyrrolidone, or combinations thereof, are excluded. The exclusion of polyethylene glycol is particularly advantageous as it is prone to form impurities on storage, especially on exposure to temperatures above 35° C.

In a preferred embodiment, salt is excluded from the aqueous ticagrelor solution for intravenous administration. The exclusion of salt is beneficial to avoid a potential salting out of ticagrelor from a cyclodextrin inclusion complex.

### Ticagrelor Solutions for Injection or Short-Term Infusion

In a preferred embodiment administration of ticagrelor is by injection or short-term infusion.

Preferably the aqueous pharmaceutical ticagrelor solution for use according to an embodiment of the invention for administration by injection or short-term infusion comprises 1-15 mg/ml ticagrelor. More preferably the solution comprises 2-14 mg/ml ticagrelor. Even more preferably the solution comprises 3-12 mg/ml ticagrelor. Most preferably the solution comprises 5-10 mg/ml ticagrelor.

Preferably the composition according to an embodiment of the invention comprises 15-40% w/w hydroxypropyl-beta-cyclodextrin. The use of this amount of cyclodextrin is advantageous for solubilization of ticagrelor and providing solutions with adequate storage stability.

Most preferably the pharmaceutical ticagrelor composition comprises 1-15 mg/ml ticagrelor and 15-40% w/w cyclodextrin.

In a preferred embodiment, the ticagrelor used for the preparation of the ticagrelor solutions of the present invention has a D90 particle size below 10 micrometers as measured using a Malvern mastersizer. It was found that a small particle size was beneficial to incorporate ticagrelor easily into cyclodextrin and thus provide solubility.

Short term infusion takes at most 30 minutes, preferably at most 25 minutes, more preferably at most 20 minutes, even more preferably at most 15 minutes.

Preferably the pharmaceutical ticagrelor composition according to an embodiment of the invention has a volume of 10-50 ml for administration by short-term infusion of at most 30 minutes.

This range of ticagrelor concentrations is adequate for use in a bacteremia treatment by intravenous administration. When the concentration is too low, unacceptable large volumes would need to be administered to provide an effective dose. This may prolong administration in an uncomfortable way. When the concentration is too high, solubility and storage stability are difficult to maintain. In addition, the product would require dilution before use.

The solutions provided above are faster acting than tablets, have a higher bio availability and can be administered to patients with difficulties to swallow or which are unconscious. Direct administration into a vein brings ticagrelor in direct contact with blood platelets. The dose is easier to adjust in function of the toxin levels that the bacterial strain concerned is releasing.

Preferably the aqueous ticagrelor composition used in the present invention is a solution consisting of:
5-15 mg/ml ticagrelor,
15-40% w/w of a hydroxypropyl-beta-cyclodextrin,
5 mM-20 mM of phosphate buffer,
wherein the pH is between 5,5 and 8.

The composition provided above is simple and easy to manufacture. The limited number of ingredients reduces the formation of impurities and side products.

It was unexpectedly found that a 5% dextrose solution is a suitable diluent compatible for dilution of the above-described concentrated aqueous solutions of a ticagrelor-cyclodextrin inclusion complex. Alternatives tested failed to provide clear ticagrelor solutions. This could be problematic for mixtures with an antibiotic formulation. The problem was solved by formulating alternative ticagrelor iv solutions suitable for administration by infusion, as described below.

### Ticagrelor Solutions for Infusion

Alternatively, to injection or short-term infusion, the intravenous administration of the aqueous ticagrelor solution is by infusion during at least 30 minutes.

Preferably the ticagrelor iv composition comprises 0,10-14 mg/ml ticagrelor and 20-100 mg/ml of cyclodextrin. Preferably the cyclodextrin is a hydroxypropyl-beta-cyclodextrin. The amount of cyclodextrin selected is a quantity for solubilization of the ticagrelor in the selected volume of aqueous pharmaceutical solution.

Preferably said aqueous pharmaceutical ticagrelor solution has a pH between 6,0-8,5 more preferably 6,5-8,0, even more preferably 6,8-7,8, most preferably around 7,5.

Preferably said aqueous pharmaceutical ticagrelor solution comprises 0,1-10.0 mg/ml ticagrelor, more preferably 0,2-8 mg/ml ticagrelor, even more preferably 0,3-6.0 mg/ml ticagrelor, most preferably 0,4-5.0 mg/ml ticagrelor or 0.5 mg-2.0 mg/ml. In a preferred embodiment, ticagrelor is the only active ingredient present in the aqueous pharmaceutical ticagrelor solution.

Preferably the ticagrelor iv composition has a volume of 25 to 1000 ml, more preferably of 50 to 750 ml, even more preferably 75 to 500 ml, most preferably 100 to 250 ml.

Unexpectedly it was found that ticagrelor can be solubilized in diluted concentrations in an aqueous medium. This is of interest in the pharmaceutical field for the treatment of ticagrelor-responsive conditions.

More preferably the aqueous pharmaceutical ticagrelor solution has a storage stability of at least 4, 5, 6, 12, 18, or 24 months as measured at 25° C. and 60% Relative Humidity.

In some embodiment, the aqueous pharmaceutical ticagrelor solution comprises a 5 w/v % dextrose solution.

Preferably said composition comprises 3000-16000 mg, more preferably 4000-15000 mg, even more preferably 5000-10000 mg, most preferably 6000-8000 mg of a cyclodextrin. That cyclodextrin is preferably a hydroxypropyl-beta-cyclodextrin. Most preferably the cyclodextrin is (2-hydroxypropyl)-beta-cyclodextrin.

### Use as a Medicine

In a preferred embodiment the bacteremia is a Staphylococcus, Streptococcus or Enterococcus bacteremia. Staphylococcus, Streptococcus and Enterococcus are all bacteria that can enter the bloodstream of a patient and cause infection.

Preferably the bacteremia is a Staphylococcus aureus bacteremia; more preferably the bacteremia is an antibiotic resistant bacterial infection; most preferably the bacteremia is a methicillin-sensitive (MSSA) or methicillin-resistant Staphylococcus aureus (MRSA). Intravenous administration of ticagrelor to a bloodstream is particularly useful for the treatment of Staphylococcus infections. This is believed to be caused by ticagrelor blocking a receptor on blood platelets present in the bloodstream, which would otherwise be attacked by alpha toxins that are produced by Staphylococcus aureus bacteria, especially by methicillin-resistant Staphylococcus aureus (MRSA). The iv-administration route provides for a prompt response to a serious systemic invasion and provide fast-acting protection of the blood platelets against bacterial attack. The inventor is of the opinion that this effect supports the survival of blood platelets. The protective effect may reduce the development and severity of thrombocytopenia in bacteremia patients. It may also prevent the development of infective (bacterial) endocarditis. Infective (bacterial) endocarditis is an infection caused by bacteria that enter the blood stream and settle in the heart lining or on a heart valve.

With the term “thrombocytopenia” as used herein in meant a lower-than-normal number of platelets (thrombocytes) in the blood. In a bacterial bloodstream infection, patients may be suffering from blood platelet loss due to bacterial attack. The blood platelet count of a healthy human is between 150.000 to 450.000 platelets per microliter of blood. In the present invention the level of 150.000 platelets per microliter of blood will be taken as threshold for thrombocytopenia.

In a preferred embodiment the aqueous ticagrelor composition for intravenous administration according to an embodiment of the invention, is administered to a patient having a blood platelet count between 50.000 and 150.000 platelets per microliter of blood (thrombocytopenia). More preferably the blood platelet count is between 60.000 and 140.000 blood platelets per microliter of blood; even more preferably between 70.000 and 130.000 blood platelets per microliter of blood; most preferably between 80.000 and 125.000 blood platelets per microliter of blood.

Administration of ticagrelor intravenously may prevent a prolonged bacteremia-derived thrombocytopenia and absence of relative increase in the blood platelet count. This may reduce mortality in bacteremia patients.

Consequently, it is also important to start the protection in the early stages of the bacterial infection when the blood platelet count would otherwise drop substantially. In a preferred embodiment of the invention, the intravenous administration of ticagrelor is initiated within 4 days of the bacterial blood stream infection. Preferably the intravenous administration of ticagrelor is initiated 4, 3, 2 or 1 day after the bacterial blood stream infection. As it may be difficult to establish the exact timing of the infection, the diagnosis of a Gram-positive blood stream infection may be taken as the reference point for initiating ticagrelor administration. A bacterial blood stream infection can be diagnosed by checking for the presence of bacteria in blood or blood culture samples.

It is also advantageous to check the alpha toxin levels that are given off by the bacterial strain concerned and to adjust ticagrelor dosing to the patient accordingly. With high alpha toxin levels, the occupation of the receptors targeted by alpha toxin, such as ADAM10 receptors targeted by alpha toxin of Staphylococcus aureus, is beneficial to reduce the impact of the bacterial infection. This protection of blood platelets may improve the clinical outcome.

In a preferred embodiment the patient is also administered an antibiotic selected from cefazolin, ceftaroline, daptomycin, ertapenem, linezolid, minocycline, oxacillin, telavancin, trimethoprim-sulfamethoxazole, vancomycin or combinations thereof. The adjunctive therapy of ticagrelor iv in conjunction with antibiotics is advantageous to provide different modes of action against the bacterial infection. Ticagrelor iv can be administered before, during or together with one or more antibiotics. For intravenous administration a small volume of aqueous ticagrelor solution, preferably 1-15 ml, may be added to an infusion solution of antibiotic.

Preferably the treatment further comprises the administration of an effective amount of blood platelets. Especially in situations where the blood platelet count has dropped drastically low, it may be beneficial to provide additional blood platelets. The addition of blood platelets serves to replace blood platelets that were lost due to bacterial attack.

For the protection of the platelet additions, pre-treatment with ticagrelor is advantageous. Preferably the blood platelets are pre-treated with ticagrelor; i.e. before administration to the patient. This avoids that the replenishment of blood platelets would only be short lived and would not be able to support the immune system in the fight against the blood stream infection.

Preferably platelet replenishment is provided by intravenous administration. Pre-treatment with ticagrelor can be obtained by injecting ticagrelor into the blood platelet solution prior to administration. Pre-treatment of blood platelets with ticagrelor has the advantage that receptors targeted by toxins of bacteria, such as ADAM10 receptors targeted by alpha toxin in Staphylococcus aureus infections, are at least partially made unavailable. This targeted (alpha) toxin-platelet interface interaction with ticagrelor may help improve the clinical outcome of a bacteremia treatment, especially in a Staphylococcus bacteremia.

In a preferred embodiment, the platelets used are prepared by freeze-drying. Freeze-drying of platelets can advantageously be obtained as disclosed in US2021100846. This product is commercially available under the tradename Thrombosomes®. The use of freeze-dried platelets is advantageous as they can be preserved for several years in dry form at ambient temperature. They can be rehydrated with sterile water within minutes for immediate infusion. In a preferred embodiment of the invention, the freeze-dried platelets are reconstituted/rehydrated with an aqueous ticagrelor solution, thus providing ticagrelor pre-treated platelets for intravenous administration.

Preferably the effective amount to be administered to the patient is less than 60 mg ticagrelor per day. More preferably the effective amount to be administered to the patient is less than 50, even more preferably less than 40, most preferably less than 30 mg ticagrelor per day.

### Ready-to-Use Ticagrelor iv

In a further aspect, the invention provides a ready-to-use aqueous solution for administration of ticagrelor by infusion, consisting of an aqueous 5% dextrose solution and an inclusion complex of ticagrelor in cyclodextrin with 1-100 mg ticagrelor and 2000-3000 mg of cyclodextrin, optionally with a buffering agent and/or a tonicity modifier.

By the term “ready-to-use” as used herein is meant a product not requiring an adjustment in composition prior to administration, such as a change in pH, osmolality, volume or ticagrelor concentration.

Preferably the ready-to-use aqueous ticagrelor solution according to an embodiment of the invention, has a storage stability of at least three months at 25° C. and 60% Relative Humidity.

Preferably the ready-to-use ticagrelor composition is provided for administration of 20-65 mg ticagrelor per day to a bacteremia patient in need thereof. Administration may be once daily. More preferably the ready-to-use composition is provided for 10-30 mg ticagrelor administration.

The invention also provides a method of preparation of a ready-to-use, aqueous ticagrelor composition for intravenous administration in a bacteremia treatment according to an embodiment of the invention. The method comprises the steps of:
providing an aqueous solution consisting of
5-15 mg/ml ticagrelor,
15-40% w/w of a hydroxypropyl-beta-cyclodextrin,
5 mM-20 mM of phosphate buffer,
optionally including a tonicity modifier,
wherein the pH is between 6,0 and 8,0;
diluting said aqueous ticagrelor solution into an aqueous 5% dextrose solution thereby obtaining said ready-to-use, aqueous ticagrelor composition for intravenous administration.

Preferably the aqueous ticagrelor solution has a volume of 10 ml and is diluted into 25 ml 5% dextrose solution.

### Packaging

In a further aspect, the invention provides a container comprising a pharmaceutical ticagrelor composition according to an embodiment of the invention. Preferably the container is a plastic bag (infusion bag) or glass bottle.

The invention provides an infusion bag comprising a ready-to-use ticagrelor solution according to an embodiment of the invention.

The invention also provides a ready-to-use container comprising 25-1000 ml of an aqueous solution, 0,10-14 mg/ml ticagrelor and 20-100 mg/ml of a cyclodextrin; preferably a hydroxypropyl-beta-cyclodextrin for use in the treatment of a Gram-positive bacteremia in a patient in need thereof by administration of the pharmaceutical composition in a therapeutically effective concentration, wherein administration is intravenously by infusion.

The invention is illustrated hereafter by the following non limiting examples.

### EXAMPLES

### Example 1

In a first example two different types of cyclodextrin were used and compared for the solubilization of ticagrelor. An unbuffered stock solution of HPBCD or SBECD was made in water at the target concentrations of 20 w/w %, 25 w/w %, 30 w/w %, 35 w/w % and 40 w/w %. Ticagrelor was added slowly under vortex. Ticagrelor was used at concentrations of 5, 10 or 14 mg/ml in milli Q water. The ticagrelor-cyclodextrin solutions were left on a shaking platform. No sonification or heat was applied.

From the results in Table 1 and 2 it follows that HPBCD was able to dissolve ticagrelor in a broader range of cyclodextrin and ticagrelor concentrations tested. Clear aqueous solutions with 5 mg/ml ticagrelor in HPBCD were obtained with 25 w/w %, 30 w/w %, 35 w/w % and 40w/w % HPBCD.

As a conclusion ticagrelor could be dissolved by leaving it on a shaking platform. No sonication was applied. HPBCD can be used at ticagrelor concentrations of 5 mg/ml, using cyclodextrin at 40% w/w, 35% w/w or 30% w/w in milliQ water. These solutions remained clear at least for the three days testing at room temperature and several days at 4° C.

TABLE 1
Solubility of ticagrelor in HPβCD

| | | Appearance of ticagrelor in cyclodextrin | | | | | |
|---|---|---|---|---|---|---|---|
| | | | After | After | | | |
| HPβCD | | After | 30 | 60 | | | |
| conc. in | Ticagrelor | 5 min | min | min | After | After | |
| MilliQ | conc. | of | of | of | 3 h of | overnight | Flocculation |
| (% w/w) | (mg/ml) | shaking | shaking | shaking | shaking | shaking | test |
| 40% | 5 | + | + | + | + | + | passed |
| | 10 | − | +/− | +/− | +/− | + | passed |
| | 14 | − | +/− | +/− | +/− | +/−* | n.a. |
| 35% | 5 | +/− | +/− | +/− | +/− | + | passed |
| | 10 | − | +/− | +/− | +/− | + | did not |
| | | | | | | | pass |
| | 14 | − | +/− | +/− | +/− | +/− | n.a. |
| 30% | 5 | − | +/− | +/− | +/− | + | passed |
| | 10 | − | − | − | − | − | n.a. |
| | 14 | − | − | − | − | − | n.a. |
| 25% | 5 | − | +/− | +/− | +/− | +* | did not |
| | | | | | | | pass |
| | 10 | − | − | − | − | − | n.a. |
| | 14 | − | − | − | − | − | n.a. |
| 20% | 5 | − | +/− | +/− | +/− | − | n.a. |
| | 10 | − | − | − | − | − | n.a. |
| | 14 | − | − | − | − | − | n.a. |

+ clear, appeared completely dissolved
− translucent solution with precipitation
+/− clear solution with precipitation
*after one hour of sonication

TABLE 2
Solubility of ticagrelor in SBECD

| | | Appearance of ticagrelor in cyclodextrin | | | | | |
|---|---|---|---|---|---|---|---|
| SBECD | | After | | | | After | |
| conc. in | Ticagrelor | 5 min | After | After | After 3 h | over- | |
| MilliQ | conc. | of | 30 min | 60 min | of | night | Flocculation |
| (% w/w) | (mg/ml) | shaking | of shaking | of shaking | shaking | shaking | test |
| 40% | 5 | − | +/− | +/− | +/− | + | passed |
| | 10 | − | − | − | − | − | n.a. |
| | 14 | − | − | − | − | − | n.a. |
| 35% | 5 | − | +/− | +/− | +/− | − | n.a. |
| | 10 | − | − | − | − | − | n.a. |
| | 14 | − | − | − | − | − | n.a. |
| 30% | 5 | − | − | − | − | − | n.a. |
| | 10 | − | − | − | − | − | n.a. |
| | 14 | − | − | − | − | − | n.a. |
| 25% | 5 | − | − | − | − | − | n.a. |
| | 10 | − | − | − | − | − | n.a. |
| | 14 | − | − | − | − | − | n.a. |
| 20% | 5 | − | − | − | − | − | n.a. |
| | 10 | − | − | − | − | − | n.a. |
| | 14 | − | − | − | − | − | n.a. |

+ clear, appeared completely dissolved
− translucent solution with precipitation
+/− clear solution with precipitation

### Example 2

After the experiments depicted in Example 1, further optimization was carried out with the selection of a suitable pH range to ensure long-term stability of the aqueous ticagrelor-cyclodextrin inclusion complex.

The following composition as provided in Table 3 was prepared.

TABLE 3
Composition for storage stability testing.

| | mg/ml |
|---|---|
| Ticagrelor | 6 |
| HPβCD 40% w/w | 452 |
| Acetate or Phosphate Buffer | Q.S to 1 ml |
| pH 4.5 to 6.5 | |

HPBCD was dissolved in a buffer solution of pH 4.5, 5.5 or 6.5 prepared separately in water. Once a clear solution was obtained, ticagrelor was dissolved in the buffer solution under constant stirring. The ticagrelor in buffer solution was filtered through a 0.22 micron filtered and filled in USP Type I glass vials. The vials were stoppered and stored. All precautions were taken during manufacturing, such as N2 purging and avoiding direct exposure to light. The vials were stored at 40° C. and 75% Relative Humidity (RH).

To determine the stability of the formulations, batches were evaluated using a related substance method on HPLC. The data of these batches is enumerated below in Table 4.

A Gradient HPLC method was used to analyze impurities in formulations using a YMC-Pack Pro C18 column (100×4.6 mm, S-3 μm 12 nm). Good separation was obtained for all the impurities.

Amine impurity: (1S,2S,3R,5S)-3-(7-amino-5-(propylsulfanyl)-3H-[1,2,3]triazolo[4,5-d]pyrimidin-3-yl)-5-(2-hydroxyethoxy) cyclopentane-1,2-diol. This is a process related degradant impurity.

Regiomer impurity: (1S, 2S, 3R, 5S)-3-((3-((1R,2S)-2-(3,4-Difluorophenyl)cyclopropyl)-5-(propylsulfanyl)-3H-[1,2,3]triazolo[4,5-d]pyrimidin-7-yl)amino)-5-(2-hydroxyethoxy) cyclopentane-1,2-diol. This is a process related degradant impurity.

Acetal impurity: 2-[[(3aR,4S,6S,6aS)-6-[7-[[1R,2S)-2-(3,4-difluorophenyl)-cyclopropyl]amino]-5-(propylsulfanyl)-3H-[1,2,3]triazolo-[4,5-d]pyrimidin-3-yl]-2,2-dimethyltetrahydro-2H-3aHcyclopenta [d][1,3]dioxol-4-yl]oxy]ethan-1-ol.

This is a process related impurity.

Triol impurity: (1S,2R,3S,4R)-4-(7-((1R,2S)-2-(3,4-difluorophenyl) cyclopropylamino)-5-(propylthio)-3H-[1,2,3]triazolo[4,5-d]pyrimidin-3-yl) cyclopentane-1,2,3-triol.

This is a process related impurity.

It was observed that only regiomer impurity increased in 4 weeks 40° C. and 75% RH at almost 0.3% level; specification limit 0.3%. Hence to optimize the stability of the product further, investigations were carried out at pH 7 to 8.

After the experiments depicted in Example 1, further optimization was carried out with the selection of a suitable pH range to ensure long-term stability of the aqueous ticagrelor-cyclodextrin inclusion complex.

The following composition as provided in Table 3 was prepared.

TABLE 3
Composition for storage stability testing.

| | mg/ml |
|---|---|
| Ticagrelor | 6 |
| HPβCD 40% w/w | 452 |
| Acetate or Phosphate Buffer | Q.S to 1 ml |
| pH 4.5 to 6.5 | |

HPβCD was dissolved in a buffer solution of pH 4.5, 5.5 or 6.5 prepared separately in water. Once a clear solution was obtained, ticagrelor was dissolved in the buffer solution under constant stirring. The ticagrelor in buffer solution was filtered through a 0.22 micron filtered and filled in USP Type I glass vials. The vials were stoppered and stored. All precautions were taken during manufacturing, such as N2 purging and avoiding direct exposure to light. The vials were stored at 40° C. and 75% Relative Humidity (RH).

To determine the stability of the formulations, batches were evaluated using a related substance method on HPLC. The data of these batches is enumerated below in Table 4.

A Gradient HPLC method was used to analyze impurities in formulations using a YMC-Pack Pro C18 column (100×4.6 mm, S-3 μm 12 nm). Good separation was obtained for all the impurities.

Amine impurity: (1S,2S,3R,5S)-3-(7-amino-5-(propylsulfanyl)-3H-[1,2,3]triazolo[4,5-d]pyrimidin-3-yl)-5-(2-hydroxyethoxy) cyclopentane-1,2-diol. This is a process related degradant impurity.

Regiomer impurity: (1S, 2S, 3R, 5S)-3-((3-((1R,2S)-2-(3,4-Difluorophenyl)cyclopropyl)-5-(propylsulfanyl)-3H-[1,2,3]triazolo[4,5-d]pyrimidin-7-yl)amino)-5-(2-hydroxyethoxy) cyclopentane-1,2-diol. This is a process related degradant impurity.

Acetal impurity: 2-[[(3aR,4S,6S,6aS)-6-[7-[[1R,2S)-2-(3,4-difluorophenyl)-cyclopropyl]amino]-5-(propylsulfanyl)-3H-[1,2,3]triazolo-[4,5-d]pyrimidin-3-yl]-2,2-dimethyltetrahydro-2H-3aHcyclopenta [d][1,3]dioxol-4-yl]oxy]ethan-1-ol.

This is a process related impurity.

Triol impurity: (1S,2R,3S,4R)-4-(7-((1R,2S)-2-(3,4-difluorophenyl) cyclopropylamino)-5-(propylthio)-3H-[1,2,3]triazolo[4,5-d]pyrimidin-3-yl) cyclopentane-1,2,3-triol.

This is a process related impurity.

It was observed that only regiomer impurity increased in 4 weeks 40° C. and 75% RH at almost 0.3% level; specification limit 0.3%. Hence to optimize the stability of the product further, investigations were carried out at pH 7 to 8.

### Example 3

Following the experiment described in example 2, a storage stability study at pH 7.5 was conducted

First HPβCD was dissolved in a phosphate buffer solution of pH 7.5 prepared separately in water. Once a clear solution was obtained, ticagrelor was dissolved in the solution under constant stirring. The solution was filtered through a 0.22 micrometer filter and filled in USP Type I amber colored glass vials. The vials were stoppered and stored. All precautions were taken during manufacturing, such as N2 purging and avoiding direct exposure to light.

TABLE 4
Stability test of ticagrelor-cyclodextrin inclusion complex in aqueous solution at pH 4.5, 5.5 and 6.5 after storage at 40° C. and 75% RH

| | | Timepoint | | | | | | | | |
|---|---|---|---|---|---|---|---|---|---|---|
| | | T = 0 | | | T = 2 weeks | | | T = 4 weeks | | |
| | | Condition | | | | | | | | |
| | | pH 4.5 | pH 5.5 | pH 6.5 | pH 4.5 | pH 5.5 | pH 6.5 | pH 4.5 | pH 5.5 | pH 6.5 |
| | | Average | Average | Average | Average | Average | Average | Average | Average | Average |
| Relative | | impurity | impurity | impurity | impurity | impurity | impurity | impurity | impurity | impurity |
| retention time | | content | content | content | content | content | content | content | content | content |
| (minutes) | Impurity | (%) | (%) | (%) | (%) | (%) | (%) | (%) | (%) | (%) |
| 0.45 | Amine | 0.05 | 0.04 | 0.05 | 0.05 | 0.05 | 0.05 | 0.05 | 0.04 | 0.05 |
| | impurity | | | | | | | | | |
| 0.70 | Oxidative | 0.00 | 0.00 | 0.00 | 0.00 | 0.00 | 0.00 | 0.01 | 0.01 | 0.00 |
| | impurity 1 | | | | | | | | | |
| 0.70 | Oxidative | 0.00 | 0.00 | 0.00 | 0.00 | 0.00 | 0.00 | 0.02 | 0.02 | 0.04 |
| | impurity 2 | | | | | | | | | |
| 0.97 | Triol | 0.05 | 0.05 | 0.06 | 0.05 | 0.05 | 0.06 | 0.05 | 0.05 | 0.05 |
| | Impurity | | | | | | | | | |
| 1.02 | Regiomer | 0.00 | 0.00 | 0.00 | 0.00 | 0.00 | 0.00 | 0.42 | 0.30 | 0.35 |
| | impurity | | | | | | | | | |
| 1.50 | Acetal | 0.05 | 0.05 | 0.06 | 0.06 | 0.05 | 0.06 | 0.05 | 0.05 | 0.06 |
| | impurity | | | | | | | | | |
| 1.52 | | 0.03 | 0.02 | 0.03 | 0.04 | 0.03 | 0.04 | 0.03 | 0.02 | 0.03 |
| Total Impurities (%) | | 0.36 | 0.33 | 0.36 | 0.40 | 0.36 | 0.41 | 0.80 | 0.65 | 0.72 |
| Sum Impurities > 0.05 | | 0.22 | 0.16 | 0.22 | 0.23 | 0.21 | 0.23 | 0.63 | 0.45 | 0.56 |
| (%) | | | | | | | | | | |

TABLE 5
Composition for storage stability testing.

| | mg/ml |
|---|---|
| Ticagrelor | 6 |
| HPβCD 40% w/w | 452 |
| Phosphate buffer pH 7.5 | Q.S to 1 ml |

TABLE 6
Storage stability study of ticagrelor-cyclodextrin inclusion complex in
aqueous solution at pH 7.5 stored at 40° C. and 75% Relative Humidity.

| Time point | | | T = 4 | T = 8 | T = 12 | T = 24 |
|---|---|---|---|---|---|---|
| 40° C. and 75% RH | | T = 0 | weeks | weeks | weeks | Weeks |
| Assay ticagrelor (%) | | 104.27 | 104.47 | 102.59 | 102.31 | 104.39 |
| Relative | Impurity ID | Impurity | Impurity | Impurity | Impurity | Impurity |
| retention | | (%): | (%): | (%): | (%): | (%): |
| time | | | | | | |
| (minutes) | | | | | | |
| 0.45 | Amine | 0.04 | 0.05 | 0.05 | 0.05 | 0.07 |
| | impurity | | | | | |
| 0.97 | Triol | 0.04 | 0.05 | 0.05 | 0.04 | 0.05 |
| | impurity | | | | | |
| 1.03 | Regiomer | 0.00 | 0.01 | 0.04 | 0.08 | 0.16 |
| | impurity | | | | | |
| 1.49 | Acetal | 0.05 | 0.05 | 0.05 | 0.05 | 0.05 |
| | impurity | | | | | |

| | | | | | |
|---|---|---|---|---|---|
| Total impurities (%) | 0.31 | 0.32 | 0.29 | 0.41 | 0.35 |
| Sum impurities > 0.05 (%) | 0.11 | 0.14 | 0.15 | 0.23 | 0.33 |

Based on the results of the stability study, as summarized in Table 6, it was concluded that good storage stability was obtained at accelerated storage conditions of 40° C. and 75% Relative Humidity. The regiomer impurity was well under control and no other impurity was of a concern.

### Example 4

In a further experiment, to optimize the concentration of HPβCD below 40% w/w, heat at 40° C. was applied at concentrations where a clear solution was difficult to obtain to help dissolve the target ticagrelor dose.

Direct physical stability data as obtained from the ticagrelor 5 mg/ml concentrate and with the flocculation test (20 μl sample material in 1 ml diluent) are shown in Table 7. Table 7 contains data on assay, purity, osmolality and pH.

TABLE 7
Physical stability of ticagrelor 5 mg/ml batches with varying HPβCD
concentrations. Data are sorted by HPβCD strength. Ticagrelor
concentration when diluted into dextrose or saline: 0.1 mg/ml.

| HPβCD | | Appearance | Appearance |
|---|---|---|---|
| strength | Appearance, | in 5% | in NaCl |
| (% w/w) | undiluted | dextrose | 0.9% |
| 32.5 | Clear solution | Clear solution | Clear solution |
| 30.0 | Clear solution | Clear solution | Turbid |
| 27.5 | Turbid; clear after heating to | Clear solution | Turbid |
| | 40° C. | | |
| 25.0 | Turbid; clear after heating to | Clear solution | Turbid |
| | 40° C. | | |
| 22.5 | Turbid; clear after heating to | Clear solution | Turbid |
| | 40° C. | | |

Because of the poor physical stability results when diluted in saline, 32.5% w/w HPβCD was chosen for a 5 mg/ml ticagrelor formulation. Undiluted concentrate remained stable even in the refrigerator with a HPβCD concentration as low as 22.5% w/w. Such a concentration yielded a nearly isotonic formulation.

In conclusion, it was possible to dissolve 5-15 mg/ml ticagrelor together with HPβCD in the concentration range of 20-40% w/w, without the use of heat. It was possible to achieve good solubility with lower concentrations of HPβCD such as 15-20% w/w with application of heat to achieve a clear solution.

At least 15% w/w HPβCD was required to provide a clear, storage stable ticagrelor solution with a concentration that is relevant for injection or intravenous administration.

### Example 5

From the results obtained in Example 4 it follows that the concentration of excipients may be such that the resulting ticagrelor solution is hypertonic. The osmolality and pH of several batches was checked. The solutions had a 19 mM phosphate buffer and pH 7.5. The results are provided in Table 8.

TABLE 8
pH and osmolality determination in undiluted batches.

| HPβCD | | Total | | Osmolality |
|---|---|---|---|---|
| strength | Assay | impurities | pH | undiluted |
| (% w/w) | (%) | (%) | undiluted | (mOsm/kg) |
| 32.5 | 99.26 | 0.41 | 7.68 | 813 |
| 30.0 | 107.64 | 0.44 | 7.74 | 638 |
| 27.5 | 104.27 | 0.42 | 7.69 | 549 |
| 25.0 | 103.23 | 0.41 | 7.69 | 493 |
| 22.5 | 98.15 | 0.39 | 7.67 | 392 |

Dilution studies were conducted to search for suitable diluents.

5 mg/ml ticagrelor-cyclodextrin solutions with varying amounts of HPβCD were diluted with normal saline, 5% dextrose solution or Ringer's lactate solution. The stability was screened. The ticagrelor concentration when diluted into dextrose or saline was 0.1 mg/ml. The results are summarized in Table 9.

TABLE 9
Diluent tests

| HPβCD | | Appearance | Appearance |
|---|---|---|---|
| strength | Appearance, | in 5% | in NaCl |
| (% w/w) | undiluted | dextrose | 0.9% |
| 32.5 | Clear solution | Clear solution | Clear solution |
| 30.0 | Clear solution | Clear solution | Turbid |
| 27.5 | Turbid; clear after heating to | Clear solution | Turbid |
| | 40° C. | | |
| 25.0 | Turbid; clear after heating to | Clear solution | Turbid |
| | 40° C. | | |
| 22.5 | Turbid; clear after heating to | Clear solution | Turbid |
| | 40° C. | | |

In addition, a screening was conducted on the impact of buffer strength on pH and osmolality. The results are summarized in Table 10.

TABLE 10
Impact of buffer strength on pH, osmolality, assay, and impurities.

| Phosphate | | | | | | |
|---|---|---|---|---|---|---|
| buffer pH | | | Osmolality | Osmolality | | Total |
| 7.5 strength | pH | pH | sample | placebo | Assay | impurities |
| (mM) | sample | placebo | (mOsm/kg) | (mOsm/kg) | (%) | (%) |
| 19 | 7.70 | 7.71 | 758 | 790 | 102.58 | 0.42 |
| 10 | 7.69 | 7.72 | 724 | 692 | 102.98 | 0.42 |
| 5 | 7.66 | 7.73 | 703 | 725 | 100.49 | 0.40 |
| 0.19 | 7.18 | 7.45 | 809 | 771 | 102.23 | 0.42 |

It was concluded that a phosphate buffer of pH 7,5 at different buffer strengths had little effect on the osmolality, except at 0.19 mM buffer strength. This buffer strength was too weak, which lead to a change in pH.

### Example 6

In a further example the impact of particle size on solubility was tested.

Two different particle size diameters for the ticagrelor active ingredient were screened, 5.5 and 15 micrometers. The pH and osmolality were not affected. Smaller particles showed a faster dissolution time, as summarized in Table 11.

The micronized ticagrelor showed significant improvement on the dissolution time. Consequently, a micronized ticagrelor with D90 of less than 10 micrometers is preferred.

With the term “D90” as used herein, is meant that at least 90% of the particles present have a size that is less than the target particle size. However, it is understood that variations in input particle size distribution (PSD) of ticagrelor would be possible and it will have an impact on the dissolution rate of ticagrelor.

TABLE 11
Impact of particle size

| | | | |
|---|---|---|---|
| Particle distribution, | Dissolution | | Osmolality |
| D90 (0.9) | time | pH | (mOsm/kg) |

| | | | | |
|---|---|---|---|---|
| 5.5 μm, micronized | 33 | min | 7.68 | 751 |
| 15 μm, unmicronized | 125 | min | 7.70 | 753 |

### Example 7

To optimize the HPβCD concentration and the pH of the solution for intravenous use, a 12 week/3 months stability study was conducted. A composition of 32,5% w/w HPβCD with 5 mg/ml ticagrelor at pH 7 to 8 was prepared and stored. Its stability was tested at regular intervals.

A comparison of stability profile at 3 different pH-7, 7.5 and 8—was carried out as below, the manufacturing process for all 3 formulations was kept constant with buffer strength at 19 mM. The results are summarized in Tables 12 to 14

TABLE 12
Storage stability in amber glass vials - pH 7

| | | Amber colour USP Type I | | | |
|---|---|---|---|---|---|
| | | 19 mM pH 7 Phosphate Buffer | | | |
| | | T0 | 1 M | 2 M | 3 M |
| | | Description | | | |
| HPβCD | 40% w/w | clear | clear | clear | clear |
| | Assay | 98.95 | 99.73 | 99.81 | 98.51 |
| | pH | 7.33 | 7.34 | 7.34 | 7.30 |
| RRT | Impurity | % | % | % | % |
| 0.45 | Amine impurity | 0.04 | 0.04 | 0.04 | 0.06 |
| 0.97 | Triol impurity | 0.05 | 0.02 | 0.04 | 0.04 |
| 1.03 | Regiomer | 0.00 | 0.03 | 0.06 | 0.11 |
| 1.49 | Acetal impurity | 0.05 | 0.05 | 0.05 | 0.05 |
| | Total | 0.41 | 0.27 | 0.34 | 0.40 |

| | | | | |
|---|---|---|---|---|
| Sum impurities >0.05 (%) | 0.22 | 0.05 | 0.11 | 0.22 |

TABLE 13
Storage stability in amber glass vials - pH 7.5

| | | Amber colour USP Type I | | | |
|---|---|---|---|---|---|
| | | 19 mM pH 7.5 Phosphate Buffer | | | |
| | | T0 | 1 M | 2 M | 3 M |
| | | Description | | | |
| HPβCD | 40% w/w | clear | clear | clear | clear |
| | Assay | 104.27 | 104.47 | 102.59 | 102.31 |
| | pH | 7.40 | 7.50 | 7.48 | 7.55 |
| RRT | Impurity | % | % | % | % |
| 0.45 | Amine impurity | 0.04 | 0.05 | 0.05 | 0.05 |
| 0.97 | Triol impurity | 0.04 | 0.05 | 0.05 | 0.04 |
| 1.03 | Regiomer | 0.00 | 0.01 | 0.04 | 0.08 |
| 1.49 | Acetal impurity | 0.05 | 0.05 | 0.05 | 0.05 |
| | Total | 0.31 | 0.32 | 0.29 | 0.41 |

| | | | | |
|---|---|---|---|---|
| Sum impurities >0.05 (%) | 0.11 | 0.14 | 0.15 | 0.23 |

TABLE 14
Storage stability in amber glass vials - pH 8.0

| | | Amber colour USP Type I | | | |
|---|---|---|---|---|---|
| | | 19 mM pH 8 Phosphate buffer | | | |
| | | T0 | 1 M | 2 M | 3 M |
| | | Description | | | |
| HPβCD | 40% w/w | clear | clear | clear | clear |
| | Assay | 103.25 | 105.88 | 105.46 | 104.46 |
| | pH | 8.07 | 8.10 | 8.04 | 8.03 |
| RRT | Impurity | % | % | % | % |
| 0.45 | Amine impurity | 0.04 | 0.05 | 0.05 | 0.07 |
| 0.97 | Triol impurity | 0.06 | 0.03 | 0.05 | 0.05 |
| 1.03 | Regiomer | 0.00 | 0.01 | 0.03 | 0.06 |
| 1.49 | Acetal impurity | 0.05 | 0.05 | 0.05 | 0.05 |
| | Total | 0.43 | 0.26 | 0.32 | 0.37 |

| | | | | |
|---|---|---|---|---|
| Sum impurities >0.05 (%) | 0.25 | 0.09 | 0.15 | 0.23 |

From the above data it was concluded that the ticagrelor solution in HPβCD was stable in the pH range of 7 to 8.

### Example 8

To study the potential impact of the packaging material on the stability of the ticagrelor-cyclodextrin inclusion complex, a composition with 32.5% w/w HPβCD was prepared with procedures and precaution's similar to previous trials; samples were stored in transparent clear glass vials and amber colored glass vials at a temperature of 40° C./75% RH. The results are shown in Table 15 and Table 16.

The results of the accelerated storage stability test indicated that after 3 months, no significant difference was observed between the two. All the samples remained clear aqueous solutions. The pH of the samples remained stable. Impurities did not change significantly.

It seems that both clear and amber colored glass vials can be used.

Compared to the results of the accelerated storage stability test on ticagrelor solutions, without the use of cyclodextrin, it is clear that the use of cyclodextrin is important to achieve a good stability. Without the cyclodextrin. 6 to 8 different impurities developed on storage. These impurities were not seen in the selected composition.

TABLE 15
Study of the potential impact of packaging.
Stability in amber color USP Type I glass.

| | | Amber colour USP Type I Glass vials | | | |
|---|---|---|---|---|---|
| | | 5 mM pH 7.5 Phosphate Buffer | | | |
| | | T0 | 1 M | 2 M | 3 M |
| | | Description | | | |
| HPβCD | 32.5% w/w | clear | clear | clear | clear |
| | Assay | 98.34 | 100.24 | 99.89 | 98.93 |
| | pH | 7.66 | 7.70 | 7.69 | 7.66 |
| RRT | Impurity | % | % | % | % |
| 0.45 | Amine impurity | 0.04 | 0.04 | 0.04 | 0.06 |
| 0.97 | Triol impurity | 0.04 | 0.02 | 0.04 | 0.05 |
| 1.03 | Regiomer | 0.00 | 0.01 | 0.02 | 0.04 |
| 1.49 | Acetal impurity | 0.05 | 0.05 | 0.04 | 0.05 |
| | Total | 0.40 | 0.22 | 0.28 | 0.33 |

| | | | | |
|---|---|---|---|---|
| Sum impurities >0.05 (%) | 0.18 | 0.05 | 0.00 | 0.16 |

TABLE 16
Study of the potential impact of packaging.
Stability clear glass vials USP Type I.

| | | Clear USP Type I Glass vials | | | |
|---|---|---|---|---|---|
| | | 5 mM pH 7.5 Phosphate buffer | | | |
| | | T0 | 1 M | 2 M | 3 M |
| | | Description | | | |
| HPβCD | 32.5% w/w | clear | clear | clear | clear |
| | Assay | 98.34 | 101.71 | 99.69 | 98.25 |
| | pH | 7.65 | 7.65 | 7.63 | 7.58 |
| RRT | Impurity | % | % | % | % |
| 0.45 | Amine impurity | 0.04 | 0.04 | 0.04 | 0.06 |
| 0.97 | Triol impurity | 0.05 | 0.03 | 0.05 | 0.05 |
| 1.03 | Regiomer | 0.00 | 0.01 | 0.02 | 0.04 |
| 1.49 | Acetal impurity | 0.05 | 0.04 | 0.04 | 0.05 |
| | Total | 0.40 | 0.22 | 0.28 | 0.33 |

| | | | | |
|---|---|---|---|---|
| Sum impurities >0.05 (%) | 0.22 | 0.00 | 0.05 | 0.16 |

Surprisingly it could be concluded that ticagrelor solutions can be stabilized with HPβCD in both amber colored and clear glass vials.

### Example 9

Further embodiments of the invention are provided as summarized in Table 17. Further improvement in achieving higher solubility of ticagrelor was tried with different concentrations, such as with 40% w/w HPβCD; a ticagrelor solubility of 13 mg/ml was also possible.

TABLE 17
clear aqueous solutions with ticagrelor-cyclodextrin
inclusion complex considering 65 mg dose.

| | | | | | | | Flocculation | |
|---|---|---|---|---|---|---|---|---|
| | HPβCD | | Final | | | | in | Flocculation |
| | (% | Ticagrelor | Vol | HPβCD | HPβCD | | saline, | in dextrose, |
| Nr | w/w) | (mg/ml) | ml | mg/ml | g/vial | Appearance | 1:2 | 1:2 |
| A | 32.5 | 7.5 | 10 | 367.25 | 3.67 | Clear, | Clear | Clear |
| | | | | | | colorless | | |
| B | 32.5 | 5 | 15 | 367.25 | 5.50 | Clear, | Clear | Clear |
| | | | | | | colorless | | |
| C | 22.5 | 5 | 15 | 254.25 | 3.81 | Clear, | Clear | Clear |
| | | | | | | colorless | | |
| C | 40 | 13 | 5 | 452 | 3.39 | Clear, | Clear | Clear |
| | | | | | | colorless | | |
| D | 33 | 8.25 | 9.2 | 372.3 | 3.43 | Clear, | Clear | Clear |
| | | | | | | colorless | | |
| E | 30 | 7.5 | 10 | 339 | 3.39 | Clear, | Clear | Clear |
| | | | | | | colorless | | |

Density 1.130 gm/cc

Based on the investigations it was observed that a concentration of 5 −13 mg/ml ticagrelor solution could be achieved using 20-40% w/w HPβCD. The volume of the fill content can be changed based on the dose required.

Surprisingly it was found that the target dose of 5-15 mg/ml ticagrelor contained in a small volume could be achieved by adjusting the HPβCD % and total available volume of the formulation ready to inject. Being able to contain the ticagrelor dose in a volume of 5-15 ml is highly relevant as it is a typical bolus injection volume.

### Example 10

In a further example, the maximum solubility of ticagrelor in an HPβCD solution, without the use of heat, was investigated. The results are summarized in Table 18.

Depending on the amount of ticagrelor to be delivered to a patient and the restriction of the sample volume as determined by an administration by injection or infusion, it follows that to dissolve 65-75 mg ticagrelor an amount of 2000-4000 mg of HPβCD per vial of 10 ml may be required.

TABLE 18
Concentration of HPBCD, dose and volume of formulations

| HPβCD | HPβCD | mg/ml | Ticagrelor | 75 mg | HPβCD | 65 mg | HPβCD |
|---|---|---|---|---|---|---|---|
| % w/w | % w/v | HPβCD | mg/ml | dose | mg/vial | dose | mg/vial |
| 17 | 19.21 | 192.10 | 4 | 18.75 | 3600 | 16.25 | 3121 |
| | | | | ml | | ml | |
| 22.5 | 25.42 | 254.20 | 5 | 15.00 | 3813 | 13 | 3304 |
| | | | | ml | | ml | |
| 30 | 33.90 | 339.00 | 8 | 9.37 | 3176 | 8.12 | 2752 |
| | | | | ml | | ml | |
| 33 | 37.29 | 372.90 | 9 | 8.33 | 3107 | 7.22 | 2692 |
| | | | | ml | | ml | |
| 40 | 45.2 | 452.00 | 13 | 7.76 | 3507 | 5 | 2260 |
| | | | | ml | | ml | |

Density of the HPβCD solution 1.130 gm/cc

Surprisingly the solutions provided in Table 18 were compatible with diluents to provide infusions, specifically with dextrose 5% in water.

### Example 11

In another embodiment of this invention, a highly stable clear solution of ticagrelor could be obtained by applying appropriate heat to the solution during preparation, thus providing a completely clear solution of the formulation at desired HPβCD and ticagrelor concentrations.

To investigate the impact of temperature and hold time, a new composition was prepared as per below Table 19.

TABLE 19
Composition for temperature impact assessment.

| | mg/ml |
|---|---|
| Ticagrelor | 8 |
| HPβCD 30% w/w | 329 |
| Phosphate Buffer | Q.S to 1 ml |
| pH 7.5 in water | |

In first step a phosphate buffer at pH 7.5 was prepared and the buffered solution was heated to 40° C.-45° C. HPβCD was added to the buffered solution under continuous mixing. Once a clear solution was obtained, ticagrelor was dispersed into the HPβCD solution and mixed until a clear solution was obtained. It usually took 30 mins to 4 hours depending on batch size. Then this solution was filtered through a 0.22-micron filter and packed in suitable clear or amber colored glass vials.

TABLE 20
Bulk hold study at 45° C.

| | | Time points @ 45° C.- Bulk | |
|---|---|---|---|
| | | hold in Glass vials | |
| | | T = 1 h | T = 110 h |
| Assay ticagrelor (%) | | 107.87 | 109.10 |
| Relative | Impurity | Average content | |
| retention time: | | impurity (%): | |
| 0.45 | Amine impurity | 0.05 | 0.07 |
| 0.97 | Triol impurity | 0.04 | 0.05 |
| 1.33 | | 0.07 | 0.03 |
| 1.47 | Acetal impurity | 0.04 | 0.04 |

| | | |
|---|---|---|
| Total impurities (%) | 0.20 | 0.28 |
| Sum impurities >0.05 (%) | 0.12 | 0.11 |

TABLE 21
Bulk Hold study at 25° C. and 40° C.

| Bulk solution @ | Ticagrelor 8 mg/ml in 30% w/w HPβCD, | | |
|---|---|---|---|
| sealed vials | phosphate buffer pH 7.5, 25° C. and 40° C. | | |
| | | Time point | |
| | | T = 1 M, | T = 1 M, |
| | | 25° C. | 40° C. |
| Assay ticagrelor (%) | | 107.22 | 105.16 |
| Relative | Impurity | Average content | |
| retention time: | | impurity (%): | |
| 0.13 | Amine impurity | 0.06 | 0.06 |
| 0.96 | Triol impurity | 0.01 | 0.00 |
| 1.03 | | 0.02 | 0.02 |
| 1.07 | Regiomer impurity | 0.03 | 0.08 |
| 1.33 | | 0.03 | 0.08 |
| 1.78 | Acetal impurity | 0.03 | 0.00 |

| | | |
|---|---|---|
| Total impurities (%) | 0.23 | 0.28 |
| Sum impurities >0.05 (%) | 0.06 | 0.21 |

TABLE 22
Bulk hold at 30° C.

Time point

| | T0 | T4 W |
|---|---|---|
| Assay ticagrelor (%) | 105.60 | 106.14 |

| | | |
|---|---|---|
| Relative | Impurity | Average content |
| retention time: | | impurity (%): |

| | | | |
|---|---|---|---|
| 0.45 | Amine impurity | 0.06 | 0.06 |
| 0.98 | Triol impurity | 0.05 | 0.04 |
| 1.06 | Regiomer impurity | 0.00 | 0.00 |
| 1.50 | Acetal impurity | 0.04 | 0.04 |

| | | |
|---|---|---|
| Total impurities (%) | 0.21 | 0.21 |
| Sum impurities >0.05 (%) | 0.10 | 0.06 |

The hold time study at temperatures between 25° C.-45° C. indicated how even 30% w/w HPβCD was capable of stabilizing ticagrelor, even after heating the solution for a prolonged period of time or keeping the bulk at an elevated temperature.

### Ready-to-Use Formulations

### Example 13

With the aim to formulate a ready-to-use ticagrelor aqueous composition, several diluted ticagrelor compositions were made and tested for solubility and stability.

TABLE 24
ready to use ticagrelor compositions in water

| | RTU Infusion | RTU Infusion | RTU Infusion |
|---|---|---|---|
| | Formulation | Formulation | Formulation |
| | mg/bottle | mg/bottle | mg/bottle |
| | 0.65 mg/ml | 0.325 mg/ml | 0.10 mg/ml |
| | TCG | TCG | TCG |
| | concentration | concentration | concentration |
| | Composition | Composition | Composition |
| | 13.1 | 13.2 | 13.3 |
| Ticagrelor | 65 | 65 | 65 |
| Hydroxy Propyl | 8000 | 16000 | 16000 |
| beta cyclodextrin | | | |
| (HPβCD) | | | |
| Water | 100 ml | 200 ml | 650 ml |
| pH | 7.33 | 7.40 | 7.57 |
| Osmolarity | 96 | 90 | 22 |
| (mOsmol/kg) | | | |
| Clarity | Clear solution | Clear solution | Clear solution |

TABLE 25
ready to use ticagrelor compositions in aqueous phosphate buffer

| | RTU Infusion | RTU Infusion | RTU Infusion |
|---|---|---|---|
| | Formulation | Formulation | Formulation |
| | mg/bottle | mg/bottle | mg/bottle |
| | 0.65 mg/ml | 0.325 mg/ml | 0.10 mg/ml |
| | TCG | TCG | TCG |
| | concentration | concentration | concentration |
| | Composition | Composition | Composition |
| | 13.4 | 13.5 | 13.6 |
| Ticagrelor | 65 | 65 | 65 |
| Hydroxy Propyl | 8000 | 16000 | 16000 |
| beta cyclodextrin | | | |
| (HPβCD) | | | |
| Phosphate buffer | 100 ml | 200 ml | 650 ml |
| pH 7.5 (10 mM | | | |
| buffer) | | | |
| pH | 7.58 | 7.45 | 7.6 |
| Osmolarity | 105 | 220 | 280 |
| (mOsmol/kg) | | | |
| Clarity | Clear solution | Clear solution | Clear solution |

TABLE 26
ready to use ticagrelor compositions in diluted saline water

| | RTU Infusion | RTU Infusion | RTU Infusion |
|---|---|---|---|
| | Formulation | Formulation | Formulation |
| | mg/bottle | mg/bottle | mg/bottle |
| | 0.65 mg/ml | 0.325 mg/ml | 0.10 mg/ml |
| | TCG | TCG | TCG |
| | concentration | concentration | concentration |
| | Composition | Composition | Composition |
| | 13.7 | 13.8 | 13.9 |
| Ticagrelor | 65 | 65 | 65 |
| Hydroxy Propyl | 8000 | 16000 | 16000 |
| beta cyclodextrin | | | |
| (HPβCD) | | | |
| Normal Saline | 100 ml | 200 ml | 650 ml |
| (0.9 w/v % Nacl | | | |
| in water) | | | |
| pH | 6.57 | 6.6 | 6.90 |
| Osmolarity | 518 | 415 | 314 |
| (mOsmol/kg) | | | |
| Clarity | Clear solution | Clear solution | Clear solution |

TABLE 27
ready to use ticagrelor compositions in dextrose solution

| | RTU Infusion | RTU Infusion | RTU Infusion |
|---|---|---|---|
| | Formulation | Formulation | Formulation |
| | mg/bottle | mg/bottle | mg/bottle |
| | 0.65 mg/ml | 0.325 mg/ml | 0.10 mg/ml |
| | TCG | TCG | TCG |
| | concentration | concentration | concentration |
| | Composition | Composition | Composition |
| | 13.10 | 13.11 | 13.12 |
| Ticagrelor | 65 | 65 | 65 |
| Hydroxy Propyl | 8000 | 16000 | 16000 |
| beta cyclodextrin | | | |
| (HPβCD) | | | |
| Dextrose 5 | 100 ml | 200 ml | 650 ml |
| w/v % | | | |
| pH | 5.12 | 5.6 | 6.61 |
| Osmolarity | 512 | 425 | 336 |
| (mOsmol/kg) | | | |
| Clarity | Clear solution | Clear solution | Clear solution |

TABLE 28
most preferred ticagrelor iv composition
for use in the present invention

| | RTU Infusion |
|---|---|
| | Formulation |
| | mg/bottle 2 mg/ml |
| | TCG concentration |
| | Composition 13.13 |
| Ticagrelor | 65 |
| Hydroxy Propyl beta | 3000 |
| cyclodextrin (HPβCD) | |
| Dextrose 5 w/v % | 30 ml |
| pH | 7.05 |
| Osmolarity | 674 |
| (mOsmol/kg) | |
| Clarity | Clear solution |

The composition has a storage stability of at least 3 months at 25° C. and 60% Relative Humidity.

### Manufacturing Process for a Ready-to-Use Infusion Formulation

A manufacturing process to make the exemplified ready-to-use solutions was as follows. In all cases a solvent as mentioned is prepared and taken in a beaker and heated to 40° C., then HPβCD is added to obtain a clear solution under stirring. After this the active ingredient ticagrelor is added at 40° C. under constant stirring until a clear solution is obtained. This solution is filtered through a 0.22-micron filter and filled aseptically in a sterile glass bottle or an infusion bag. 24 mg/ml to 350 mg/ml HPβCD was required to obtain a stable ticagrelor solution ready for infusion. The amount of cyclodextrin required was depending upon the volume of the targeted infusion medium.

Ticagrelor is an active ingredient that is insoluble in water. The more it is in a diluted aqueous solution, the more tendency it has to precipitate. A proportional increase in cyclodextrin was required as the dilution factor for ticagrelor increased, when going from 30 ml to 100 ml to 200 ml. However, for a 650 ml volume and higher an amount of 16 g of cyclodextrin was found sufficient to hold the ticagrelor in the aqueous solution.

Note that no organic co-solvent, surfactant or other solubilizer were used.

Alternative Preparation Method Starting from a Concentrated Ticagrelor Solution

It is possible to dilute 1 vial of 8 ml containing 65 mg/vial ticagrelor and about 3 g HPβCD with 25 ml 5 w/v % dextrose and obtain a clear solution with a final volume of 33 ml.

However, this was not possible with a 0,9 w/v % NaCl solution as diluent for the concentrated ticagrelor solution.

This is of importance in medical treatments where a concentrated ticagrelor aqueous composition would be mixed with another medicine. It can lead to precipitation of ticagrelor, rendering the combination product unsuitable for intravenous administration.

### Example 14-Aqueous Ticagrelor Solutions for Intravenous Administration in a Bacteremia Treatment

Aqueous ticagrelor solutions for intravenous administration were prepared with the following composition:
5-15 mg/ml Ticagrelor
20-40% w/w HPβCD
Q·S to 1 ml Acetate or Phosphate Buffer pH 4.5 to 6.5
pH solution: 6-8
Osmolality solution: 350-900 mOsm/kg

Packaged in a glass vial, containing 5-15 ml aqueous ticagrelor solution.

Storage stability: at least 3 months at 40° C. and 75% Relative Humidity

Human patients are diagnosed with a MRSA bacteremia in hospital, based on blood cultures.

Within 4 days from obtaining the positive test result, the patient is intravenously administered a first 30 mg ticagrelor in the form of an aqueous iv solution. The administration is repeated twice daily for multiple days until the bacteremia clears.

### Example 15-Aqueous Ticagrelor Solutions for Intravenous Administration in a Bacteremia and Bacteremia-Related Thrombocytopenia Treatment

Human patients are diagnosed with a MRSA bacteremia and bacteremia-related thrombocytopenia, based on blood cultures and blood platelet counts.

The patients are intravenously administered a ready-to-use aqueous ticagrelor composition as provided in Table 28, with a storage stability of at least 3 months at 25° C. and 60% Relative Humidity in a therapeutically effective amount. The administration is repeated until the bacteremia clears, and the thrombocytopenia is resolved.

### Example 16-Aqueous Ticagrelor Solutions for Intravenous Administration in a Bacteremia Treatment

Human patients are diagnosed with a Gram-positive bacteremia. Following the diagnoses, the patients are intravenously administered a ready-to-use ticagrelor iv composition as provided in Table 28 in a therapeutically effective amount.

### Example 17-Aqueous Ticagrelor Solutions for Intravenous Administration in a Bacteremia-Related Thrombocytopenia Treatment

Human patients are diagnosed with a Gram-positive bacteremia. Patient's blood platelet counts are between 80.000 and 120.000 platelets per microliter of blood. An aqueous ticagrelor iv composition having a storage stability of at least 3 months at 25° C. and 60% Relative Humidity was mixed with an infusion bag of blood platelets. The mixture was administered by infusion to the bacteremia patients in a therapeutically effective amount. The treatment is repeated at least until bacteremia clearance and restoration of blood platelet counts to levels above 150.000 platelets per microliter of blood.

1. A method of treating a Gram-positive bacteremia infection in a patient in need thereof, comprising:
administering intravenously to the patient, by injection or infusion, a therapeutically effective amount of an aqueous ticagrelor solution, wherein the solution has a storage stability of at least 3 months at 25° C. and 60% Relative Humidity or at 40° C. and 75% Relative Humidity.

2. The method of claim 1, wherein the aqueous ticagrelor solution has a pH of 5.5 to 9.0 and an osmolality between 300-900 mOsm/kg.

3. The method of claim 1, wherein the aqueous ticagrelor solution, comprises:
a water-soluble inclusion complex of ticagrelor in a cyclodextrin.

4. The method of claim 3, wherein the cyclodextrin is hydroxypropyl-beta-cyclodextrin.

5. The method of claim 3, wherein the aqueous ticagrelor solution, comprises:
a) 1-15 mg/mL ticagrelor; and,
b) 15-40% w/w cyclodextrin.

6. The method of claim 3, wherein the aqueous ticagrelor solution, comprises:
a) 0.10-14 mg/mL ticagrelor; and,
b) 20-100 mg/mL of the cyclodextrin.

7. The method of claim 1, wherein organic co-solvents are absent.

8. The method of claim 1, wherein the volume of the solution is 1-15 mL and the solution is suitable for administration by injection.

9. The method of claim 1, wherein the volume of the solution is 10-50 mL and the solution is suitable for administration by short-term infusion of ≤30 minutes.

10. The method of claim 1, wherein the volume of the solution composition is 25-1000 mL and the solution composition is suitable for administration by short-term infusion of ≥30 minutes.

11. The method of claim 1, wherein the Gram-positive bacteremia is selected from a Staphylococcus, Streptococcus, and Enterococcus bacteremia.

12. The method of claim 1, wherein the Gram-positive bacteremia is a Staphylococcus aureus bacteremia.

13. The method of claim 1, wherein the Gram-positive bacteremia is an antibiotic-resistant bacterial infection.

14. The method of claim 1, wherein the Gram-positive bacteremia is a methicillin-sensitive (MSSA) or methicillin-resistant Staphylococcus aureus (MRSA).

15. The method of claim 1, further comprising:
administering a second component which is an antibiotic selected from:
cefazolin, ceftaroline, daptomycin, ertapenem, linezolid, minocycline, oxacillin, telavancin, trimethoprim-sulfamethoxazole, vancomycin, and combinations thereof.

16. The method of claim 1, wherein the administration of ticagrelor is initiated within 4 days of the bacterial blood stream infection.

17. The method of claim 1, wherein the dose of ticagrelor is adjusted in accordance with the alpha toxin levels produced by the Gram-positive bacterial strain present in the blood stream infection.

18. The method of claim 1, wherein the patient has thrombocytopenia, which a blood platelet count between 50,000 and 150,000 platelets per microliter of blood.

19. The method of claim 17, further comprising:
administering a therapeutically effective amount of blood platelets for the treatment of the thrombocytopenia.

20. The method of claim 19, wherein the blood platelets are pre-treated with ticagrelor.

21. The method of claim 1, wherein the solution is housed in a ready-to-use container, comprising:
25-1000 mL of the aqueous ticagrelor solution.

22. The method of claim 21, wherein the aqueous ticagrelor solution, comprises:
a) 1-15 mg/mL ticagrelor; and,
b) 15-40% w/w cyclodextrin.

23. The method of claim 21, wherein the aqueous ticagrelor solution, comprises:
a) 0.10-14 mg/mL ticagrelor; and,
b) 20-100 mg/mL of the cyclodextrin.
